(19) United States
(12) Patent Application Publication
(10) Pub. No.: US 2019/0025624 A1
(43) Pub. Date: Jan. 24, 2019

(54) TWO MODE ELECTRO-OPTIC FILTER

(71) Applicant: Kimberly-Clark Worldwide, Inc., Neenah, WI (US)

(72) Inventors: Andrej Petelin, Ljubljana (SI); Igor Musevic, Ljubljana (SI)

(21) Appl. No.: 16/067,312

(22) PCT Filed: Dec. 30, 2016
(86) PCT No.: PCT/US2016/069583

§ 371 Date: Jun. 29, 2018

Related U.S. Application Data
(60) Provisional application No. 62/273,547, filed on Dec. 31, 2015.

(51) Int. Cl. G02F 1/133; G02F 1/1347; G02F 1/1335; G02F 1/13363; A61F 9/06

(52) U.S. Cl. CPC G02F 1/13306; G02F 1/13471; G02F 1/133528; G02F 1/13363; A61F 9/062; G02F 2203/48; G02F 2413/04; G02F 2413/11; G02F 2413/06; G02F 2413/05; G02F 2001/133531; G02F 2413/14

(57) ABSTRACT

Methods and systems for an electro-optic filter that operates in two modes-a homogeneous mode and a heterogeneous mode. The homogeneous mode maintains a relatively homogeneous shading/attenuation range across specified viewing angles in both the vertical and horizontal directions. The heterogeneous mode provides for relatively homogeneous shading/attenuation range across specified viewing angles in the horizontal direction but allows for gradual and a more varying (e.g., wider) range of shading/attenuation changes across the specified viewing angles in the vertical direction, e.g., as compared to the horizontal direction.

This application claims the benefit of priority from U.S. Provisional Application No. 62/273,547 filed on 31 Dec. 2015.

### BACKGROUND

This disclosure relates to electro-optic filters.

Electro-optic filter-based devices can be used to attenuate incident light. For example, electro-optic filters are used in automatic darkening filters (ADFs) in applications such as, for example, welding masks. In such applications, electro-optic filters can include a combination of polarizers and liquid crystal cells. In response to a light sensor output triggered by incident light from a welding arc, ADF-based electro-optic filters, for example, switch between an optically transparent state (e.g., when no welding is occurring so a welder can observe the surrounding without removing the helmet) and an optically darker (e.g., eye protecting) state.

The shading effects provided by many such filters are angularly dependent. Thus the user's field of view is uneven with respect to the amount of darkening/shadings. However, it is often desirable that the filters permit wide viewing angles to give the user a full range view of the surrounding environment. The European Standards EN 379 standard sets forth certain requirements including requirements for viewing angle characteristics (along with other requirements, such as light diffusion, variations in luminous transmission, and optical quality). As such, the uneven darkening of many filters caused by angular dependence effects must be addressed to meet certain standards.

Further, typical filters are flat. Thus to increase a user's horizontal line of sight (e.g., to increase the horizontal viewing field to expand the user's peripheral vision) the filters need to be wider. In turn, this causes the masks to be wider and more bulky, which can detract from the comfort and/or usability of, for example, a welding mask.

### SUMMARY

In general, the subject matter of this specification relates to electro-optic filers having two modes of operation and/or are suitable for use in curved filter surface applications. In general, one aspect of the subject matter described in this specification can be implemented in systems that include a two-mode filter device comprising a first twisted nematic liquid crystal (LC) cell comprising first top and bottom plates bounding liquid crystal material and having a twist angle of greater than 90 degrees and configured to reverse its transmission characteristic at a threshold voltage, where the first top and bottom plates are bounded between first top and bottom polarizers; a second twisted nematic LC cell comprising second top and bottom plates bounding liquid crystal material and having a twist angle of less than 90 degrees, where the second top and bottom plates are bounded between second top and bottom polarizers; a first driver configured to apply a first voltage across the first twisted nematic LC cell; a second driver configured to apply a second voltage across the second twisted nematic LC cell; and a controller configured to cause: the first driver to apply the first voltage that is greater than the threshold voltage and the second driver to apply the second voltage that is less than, equal to or greater than the threshold voltage to operate in a homogeneous mode; and the first driver to apply the first voltage that is equal to or less than the threshold voltage and the second driver to apply the second voltage that is less than, equal to or greater than the threshold voltage to operate in a heterogeneous mode. Other embodiments of this aspect include corresponding methods.

Yet another aspect of the subject matter described in this specification can be implemented in methods that include applying a first voltage, to a first twisted nematic LC cell, that is greater than a threshold voltage and applying a second voltage, to a second twisted nematic LC cell, that is less than, equal to or greater than the threshold voltage to operate in a homogeneous mode; and applying the first voltage, to the first twisted nematic LC cell, that is equal to or less than the threshold voltage and the applying the second voltage, to the second twisted nematic LC cell, that is less than, equal to or greater than the threshold voltage to operate in a heterogeneous mode; and where the first twisted nematic LC cell comprises first top and bottom plates bounding liquid crystal material and having a twist angle of greater than 90 degrees and configured to reverse its transmission characteristic at the threshold voltage, wherein the first top and bottom plates are bounded between first top and bottom polarizers; and the second twisted nematic LC cell comprises second top and bottom plates bounding liquid crystal material and having a twist angle of less than 90 degrees, where the second top and bottom plates are bounded between second top and bottom polarizers. Other embodiments of this aspect include corresponding systems.

In some implementations, the methods and systems described herein have the following features, including where the two-mode filter device includes a plurality of curved or flat filter units, that is, a main curved or flat filter unit comprising of the first twisted nematic LC cell stacked with the second twisted nematic LC cell, and a plurality of supplementary curved or flat filter units where the supplementary filter units are arranged adjacent (side-by-side) to the main filter unit to define a one- or two-dimensionally curved (e.g. spherically or cylindrically curved) filter surface of the two-mode filter device.

The first top and bottom plates and the second top and bottom plates are negative birefringent c-plates having a nominal negative birefringence with their optical axis oriented perpendicularly to the plates. The first top and bottom plates and the second top and bottom plates comprise plastic. The first top and bottom plates and the second top and bottom plates comprise glass and where one or a plurality of additional birefringent layers that are negative birefringent c-plates, with optical axis aligned along the plates normal and with a negative birefringence, are between the first top polarizer and first top plate and between the first bottom plate and first bottom polarizer. A total absolute value of the out-of-plane retardation introduced by the birefringent layers and the polarizers is between 200 and 400 nm for the first twisted nematic LC cell and less than 300 nm for the second twisted nematic LC cell.

The twist angle of the first twisted nematic LC cell is in the range of 100 to 140 degrees and the twist angle of the second twisted nematic LC cell is in the range of 60 to 80 degrees. The transmission characteristic is a transmission gradient in a vertical plane which is along the vertical symmetry axis.

The homogenous mode defines homogeneous attenuation in the horizontal and vertical planes, and where homogeneous attention in the vertical plane specifies a ratio of the luminous transmittance values measured for any angle of incidence up to +−15 degrees in a vertical direction and the transmittance value at normal incidence (or its reciprocal, whichever is greater) is less than 7.20 and homogeneous attention in the horizontal plane specifies a ratio of the luminous transmittance values measured for any angle of incidence up to +−15 in a horizontal direction and the transmittance value at normal incidence (or its reciprocal, whichever is greater) is less than 2.68.

The heterogeneous mode defines the homogeneous attenuation in the horizontal plane and inhomogeneous attenuation in the vertical plane, where inhomogeneous attenuation in the vertical plane specifies ratio of the luminous transmittance values measured for any angle of incidence up to +−15 in a vertical direction and the transmittance value at normal incidence (or its reciprocal, whichever is greater) is less than 19.31.

The first twisted nematic LC cell has polarizer transmission axes of the first top and bottom polarizers that are substantially parallel to LC alignment directions of the respective first top and bottom plates and the second twisted nematic LC cell has polarizer transmission axes of the second top and bottom polarizers that are substantially parallel to LC alignment directions of the respective second top and bottom plates.

The first twisted nematic LC cell has polarizer transmission axes of the first top and bottom polarizers that are substantially parallel to LC alignment directions of the respective first top and bottom plates and the second twisted nematic LC cell has polarizer transmission axes of the second top and bottom polarizers that are substantially perpendicular to LC alignment directions of the respective second top and bottom plates.

Particular embodiments of the subject matter described in this specification can be implemented so as to realize one or more of the following advantages. A curved (e.g. cylindrically curved) filter surface can be made with a wide viewing angle (e.g., that meets EN 379 standards), based on the construction and/or grouping of the LC cells, to expand, for example, the line of sight for a welding mask user, as compared to flat filters which would require increasing the width of the filter and therefore the width and bulk of the mask to attempt to increase the line of sight.

The filter can work in a homogeneous mode described by having a wide viewing angle that is homogeneous across its vertical and horizontal range (e.g., a ratio of the luminous transmittance values measured for any angle of incidence up to +−15 and the transmittance value at normal incidence (or its reciprocal, whichever is greater) is less than 7.20 in a vertical direction and less than 2.68 in a horizontal direction) such that the level of light attenuation (and the user's perception of such) is constant or slightly varies across the user's field of view. This provides a reduced angular dependence filter that meets, for example, class 2 requirements for welding masks.

The same filter can also work in a gradual mode (e.g., heterogeneous mode) described by having a wide viewing angle that is homogeneous (e.g., as described above) across its horizontal range and a viewing angle attenuation dependence that gradually changes across the vertical direction (e.g., a ratio of the luminous transmittance values measured for any angle of incidence up to +−15 in a vertical direction and the transmittance value at normal incidence (or its reciprocal, whichever is greater) is less than 19.31.). Such a gradual change across the vertical direction allows a user to adjust the brightness of the filter by tilting the user's head (and therefore filter) in the vertical plane. This allows the intensity of the welding light seen by the user to vary with head position/tilt, as opposed to requiring the user to constantly adjust the brightness level of the filter by, for example, adjusting a tuning/brightness controller (e.g., knob) by hand.

The details of one or more implementations of the subject matter described in this specification are set forth in the accompanying drawings and the description below. Other features, aspects, and advantages of the subject matter will become apparent from the description, the drawings, and the claims.

### DESCRIPTION OF DRAWINGS

FIG. 1 is a block diagram of an example twisted nematic liquid crystal cell based filter.

FIG. 2A is a block diagram of an example two-mode filter having a low twist LC cell and high twist LC cell.

FIG. 2B is a representation of the orientation relationships of the low twist LC cell of FIG. 2A.

FIG. 2C is a representation of the orientation relationships of the high twist LC cell of FIG. 2A.

FIG. 3A is a block diagram of another example two-mode filter having a low twist LC cell and high twist LC cell.

FIG. 3B is a representation of the orientation relationships of the low twist LC cell of FIG. 3A.

FIG. 3C is a representation of the orientation relationships of the high twist LC cell of FIG. 3A.

FIG. 4 is a representation of attenuation/shade-voltage curves of a high twist cell, a low twist cell and a two-cell filter of the low and high twist cells.

FIG. 5 is a representation of attenuation/shade-viewing angle curves of the high twist LC cell of the two mode filter.

FIG. 6 is a representation of viewing angle properties of the two mode filter where the low twist and high twist cells are symmetrically compensated with negative C retarders.

FIG. 7 is a flow chart of an example process for controlling a two-mode filter.

FIG. 8A is a block diagram of an example two-mode filter having a first high twist LC cell and a second high twist LC cell.

FIG. 8B is a representation of the orientation relationships of the first high twist LC cell of FIG. 8A.

FIG. 8C is a representation of the orientation relationships of the second high twist LC cell of FIG. 8A.

FIG. 9A is a block diagram representation of a two-mode filter with the first high twist cell and the second high twist cell symmetrically compensated with a stretched negative C retarder.

FIG. 9B is a representation of the orientation relationships of the first high twist LC cell of FIG. 9A.

FIG. 9C is a representation of the orientation relationships of the second high twist LC cell of FIG. 9A.

FIG. 9D is a representation of attenuation/shade-voltage curve of a two-cell filter of the two high twist cells of FIG. 9A.

FIG. 10 is a representation of viewing angle properties of the two mode filter with the two high twist cells symmetrically compensated with a single stretched negative C retarder.

FIGS. 11A and 11B are representations of a cylindrically curved filter surface.

Like reference symbols in the various drawings indicate like elements.

### DETAILED DESCRIPTION

The present disclosure generally relates to electro-optic filters, for example, as used in ADFs for welding masks. The electro-optic filters of the present disclosure can operate in two modes—a homogeneous mode and a heterogeneous mode (also referred to as the gradual mode). The homogeneous mode, as described above, maintains a relatively homogeneous shading/attenuation range across specified viewing angles in both the vertical and horizontal directions. The heterogeneous mode provides for relatively homogeneous shading/attenuation range across specified viewing angles in the horizontal direction but allows for gradual and a more varying (e.g., wider) range of shading/attenuation changes across the specified viewing angles in the vertical direction, e.g., as compared to the horizontal direction. Such gradual vertical shading change allows, for example, a welder using a welding helmet with the two-mode filter to select the heterogeneous mode and control the level of vertical shading by simply tilting (e.g., up or down in the vertical direction) the welding mask.

The electro-optic filters of the present disclosure can also be made curved (e.g., individual filter may or may not be curved) and/or arranged in an offset, side-by-side configuration to form a one- or two-dimensionally curved (e.g. cylindrically or spherically) surface for use in, for example, in curved ADFs (e.g., through a single or multiple filters) in welding masks, which provide the viewing benefits described above.

The electro-optic filters can be constructed from particular combinations of specific twisted nematic liquid crystal (LC) cells (LC). In some implementations, the electro-optic filter is formed from a low twist cell (e.g., having a twist angle of less than 90 degrees) stacked with a high twist cell (e.g., having a twist angle of greater than 90 degrees). To operate in the a homogeneous mode, a controller controls a first LC driver circuit to apply a voltage across the high twist LC cell that is greater than the threshold voltage of the high twist LC cell and controls a second LC driver circuit to apply a second voltage across the low twist LC cell in the operable range of the low twist LC cell (e.g., a voltage that is less than, equal to or greater than the threshold voltage). The voltage applied across the low twist cell can be used to control the filter attenuation.

The threshold voltage of the high twist LC cell defines a voltage at which the high twist LC cell reverses its transmission characteristic (e.g., a transmission gradient in a vertical plane that is along the vertical symmetry axis). For example, the threshold voltage delineates between voltage regions that cause the high twist LC cell to increase or decrease its attenuation (or shading) effects as a function of viewing angle in the vertical plane. This allows, for example, the intensity of the welding light seen by a welder using an ADF with this technology to vary with vertical head tilt.

To operate in a heterogeneous mode, the controller controls the first LC driver circuit to apply the first voltage across the high twist LC cell that is equal to or less than the threshold voltage and control the second LC driver circuit to apply the second voltage across the low twist LC cell in the operable range of the low twist LC cell (e.g., a voltage that is less than, equal to or greater than the threshold voltage). Thus by varying the voltages applied across the cells, these two-mode filters can be made to operate in either the homogeneous or heterogeneous modes.

In some implementations, the two-mode filter is formed from two vertically stacked high twist LC cells (e.g., each with twist angles of between 120-180 degrees). The amount of in-plane (e.g., RO) retardation is tuned to compensate the residual birefringence of the high-twist LC cell. Both high twist LC cells have the gradient-flipping property (e.g., transmission characteristic reversing) described above. In these implementations, the high twist LC cells are driven at voltages above their threshold voltage to operate in the homogeneous mode. In the heterogeneous mode, one of the high twist LC cells is driven at a low voltage (e.g., below the threshold voltage) and the other at a high voltage (e.g., above the threshold voltage). The direction of the gradient (whether it is darker in the upper or lower vertical viewing angle) can be flipped in this mode, depending on which of the two LC cells is driven at the low voltage.

In general, if liquid crystal is confined between two glass (or plastic) plates coated with transparent electrodes (e.g. ITO, PEDOT:PSS, Graphene, Ag nanowires, etc) and with an alignment layer, then it is aligned along the orientation direction of each alignment surface. The alignment surface and transparent electrode can be the same material (e.g. a conductive polymer). In a Twisted nematic (TN) mode, liquid crystal is mixed with a chiral dopant, which promotes the handedness and the amount of helical twist that LC molecules undergo. The alignment layer can be a rubbed polyimide (or a conductive polymer) layer, where the rubbing direction determines the orientation of the LC molecules and the tilt angle near the surface. The alignment layer can be a polymer oriented by photo-alignment techniques, where the alignment and tilt are induced by polarization of light instead of rubbing. When substrate LC alignment directions on both substrates are rotated by a certain amount with respect to each other, LC molecules tend to form a twisted structure. In 90 degree twist cells, a very small amount of chiral dopant is generally added to introduce the twist to the system. If certain optical properties of the LC material (e.g., the refractive indices) are selected to be in specific ranges, such twisted cell operates as a polarization rotator for light with a given wavelength. When placed between two crossed polarizers, the TN cell transmits light. When voltage is applied to the electrodes, LC molecules tend to orient along the surface normal and the light transmission through the cell greatly reduces or goes to zero. When the voltage is removed then the LC molecules reorient to allow light to pass.

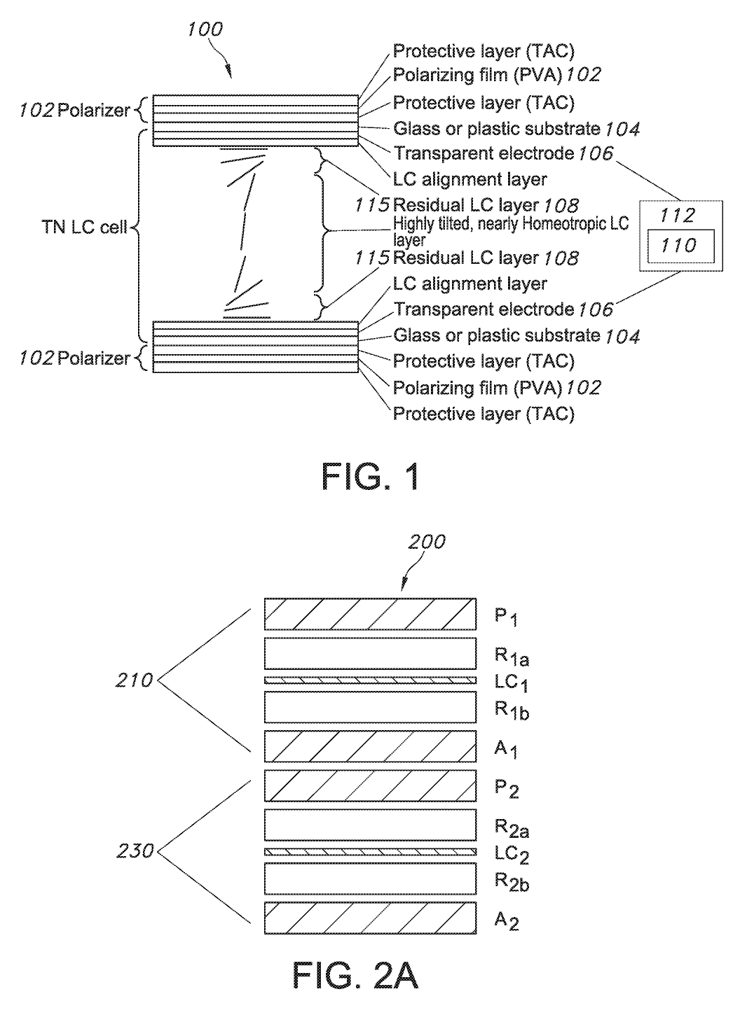

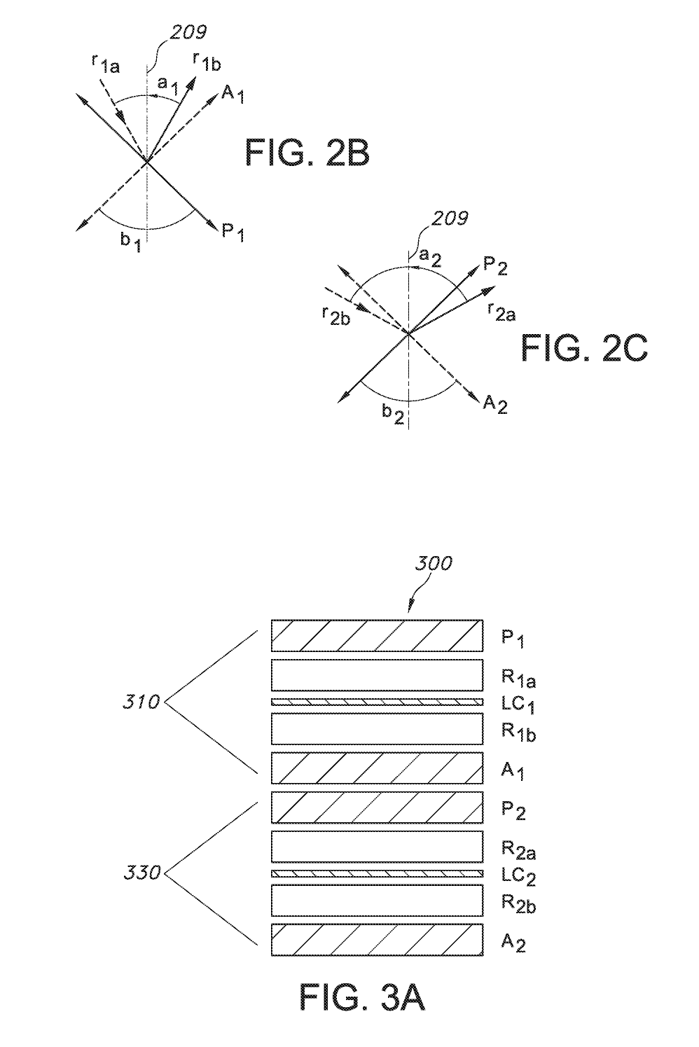

FIG. 1 is a block diagram of an example twisted nematic liquid crystal cell based filter 100. The filter 100 includes, for example and among others, polarizers 102, substrates or plates 104, electrodes 106, liquid crystal molecules (e.g., liquid crystal layer) 108, a variable voltage driver 110 and a controller 112. In some implementations, the plates 104 include the electrodes 106. The polarizers 102 are crossed (or generally crossed such as within +−10 degrees of being crossed). For example, for some twisted cells the polarizers are oriented 90 degrees apart. In some implementations, a bisector of polarization orientations of the first top and first bottom polarizers (e.g., from a high-twist LC cell of the filter 200, as described below) and a bisector of polarization orientations of the second top and bottom polarizers (e.g., from a low-twist LC cell of the filter 200, as described below) are aligned along a vertical symmetry axis (e.g., 209 in FIGS. 2B and 2C) and LC alignment layers are oriented symmetrically with respect to the vertical symmetry axis. In some implementations, plastic or glass can be used as the plates 104. In some implementations, the plates 104 are negative birefringent c-plates having a nominal (or no) negative birefringence with their optic axis oriented perpendicularly to the plates.

The liquid crystals (LC) molecules 108, as described above, can be doped with chiral dopant to promote the handedness and the amount of helical twist that LC molecules 108 undergo. The variable voltage driver 110 couples to the electrodes 106 to apply a voltage across the electrodes 106, for example, to reorient the LC molecules 108 to allow light to pass through the filter 100. The variable voltage driver 110 includes an electrical circuit that can generate voltages across a specified range and apply voltages from that range across the electrodes 106.

The controller 112 controls the driver 110 and instructs the driver 110 when and how long) to activate and apply a voltage and what voltage to apply (e.g., a specific voltage from the range). The controller 112 can be preprogramed with instructions (e.g., by a user) that specify how to control the driver 110 in response to certain inputs, e.g., a user adjustable setting to select the homogeneous or heterogeneous modes or levels of shading within each mode. Filter attenuation values can be measured in shades, which is defined as shade=1+7/3 log₁₀ (attenuation).

The homogenous mode defines homogeneous attenuation in the horizontal and vertical planes, where homogeneous attention in the vertical plane specifies a ratio of the luminous transmittance values measured for any angle of incidence up to +−15 degrees in a vertical direction and the transmittance value at normal incidence (or its reciprocal, whichever is greater) is less than 7.20 (2 shades) and homogeneous attention in the horizontal plane specifies a ratio of the luminous transmittance values measured for any angle of incidence up to +−15 degrees in a horizontal direction and the transmittance value at normal incidence (or its reciprocal, whichever is greater) is less than 2.68 (1 shade). The heterogeneous mode defines the homogeneous attenuation in the horizontal plane and inhomogeneous attenuation in the vertical plane, where inhomogeneous attenuation in the vertical plane specifies a ratio of the luminous transmittance values measured for any angle of incidence up to +−15 degrees in a vertical direction and the transmittance value at normal incidence (or its reciprocal, whichever is greater) is less than 19.31. (3 shades). Furthermore, this ratio measured for any angle between +−15 and +−30 degrees shall not be greater than 138.95 (5 shades) and a ratio between a maximum transmission value and minimum transmission value measured at angles between −15 degrees and +15 degrees in the vertical direction is in some implementations greater than 19.31 (3 shades) or in some implementations greater than or similar to 138.95 (5 shades). This way the heterogeneous mode complies with the standard (e.g. EN379), albeit having a strong variations in luminous transmittances. FIG. 1 includes parenthetical examples of specific types of layers (e.g., TAC for the protective layer); however, such layers are not so limited.

FIG. 2A is a block diagram of an example two-mode filter 200 having a low twist LC cell 210 (e.g., second LC cell) and high twist LC cell 230 (e.g. first LC cell). FIG. 2B is a representation of the orientation relationships of the low twist LC cell 210 of FIG. 2A, and FIG. 2C is a representation of the orientation relationships of the high twist LC cell 230 of FIG. 2A. The filter 200 (e.g., based on two of the filters 100, one being low twist and the other being high twist) can operate in the homogeneous mode or the heterogeneous mode, and switch between such modes, for example, based on user inputs or preprogrammed instructions.

In some implementations, the high twist cell 230 (e.g., based on filter 100) has a twist angle of around 120 degrees and, for example and optionally, is in the extraordinary mode (see paragraph 60). In some implementations, the high twist angle is between 100 and 140 degrees. The low twist cell 210 can have a twist angle of around 70 and, for example, is in either the ordinary mode or the extraordinary mode (see paragraph 60). In some implementations, the low twist angle is between 50 and 80 degrees.

The LC alignment directions (or polyimide rubbing directions) on the low twist cell 210 (e.g., based on filter 100) are denoted as r₁ₐ and r_{1b} for the top and bottom surface, respectively. The LC alignment directions (or polyimide rubbing directions) for the high twist cell 230 are denoted as r₂ₐ and r_{2b}. The LC alignment and polarizer orientations are placed symmetric with respect to the vertical symmetry axis, which is formed between the bisector between the two crossed polarizers P₁ and A₁ of the low twist cell 210 and aligned along the bisector between the two crossed polarizers P₂ and A₂ of the high twist cell 230. In some implementations, each of the cells 210, 230 has two mutually perpendicular polarizers. Cells LC alignment direction should be aligned symmetric with respect to the bisector of the two polarizers (e.g., Pₓ and A_{y}). Between these two polarizers there are two equal negative C retarders (e.g., R₁ₐ, R_{1b}, R₂ₐ, R_{2b}) such as, for example, plastic LC cell substrates 104, polarizer protective layers, and any additional layers with a combined negative c retardation properties. The angle between the two crossed polarizer pairs is denoted as b₁ and b₂ for the first and second cells 210, 230, respectively. Both such angles are at 90 degrees. In some implementations, these angles can vary up to +−10 degrees from 90 degrees.

As described above, in some implementations, there are four negative C retarders placed in the filter 200, R₁ₐ, R_{1b}, R₂ₐ, R_{2b}, and the out-of-plane retardation of each of the retarders is between 50 and 150 nm (e.g., depending on the intrinsic retardation of the TAC layer found in the polarizer). In some implementations, a plastic substrate (e.g., 104) can take the role of (phase) retarders R₁ₐ, R_{1b}, R₂ₐ, and R_{2b} (e.g., acts a simple negative C retarder, with no A-plate retardation). However, in some implementations, if glass substrates (e.g., 104) are used, only a single retarder (e.g., but with double retardation value) can be used on each of the cells 210, 230. In some implementations, if glass substrates (e.g., 104) are used, there are no additional retarders placed between the polarizer and glass in cell 210.

In some implementations, the twist angles a₁ and a₂ are set so that the mean twist of both cells 210, 230 remains around 90 degrees (e.g., 60 and 120 twist) so that the offset angle between the LC alignment directions r₁ₐ and r₂ₐ and the polarizers P₁ and P₂ remain approximately the same (e.g., +−10 percent), but in the opposite direction. The low twist and high twist configuration result in a residual positive in-plane birefringence which lowers the contrast ratio of the filter 200 and modifies the viewing angle properties. In some implementations, viewing angle in the vertical direction increases with the difference between the high twist and low twist angles, but so does the driving voltage. If the difference between the high twist and low twist angles is too low, for example, it may be difficult to operate the filter 200 in the homogeneous mode at low shade values (e.g., shade 9, for instance).

In some implementations, in the homogeneous mode, both cells 210, 230 can be driven (e.g., by separate drivers 110 or the same driver 110) at a high voltage in a low slope area of a transmission voltage curve. This low slope area is more apparent in high twist cells and is characterized by a lower slope of the transmission-voltage curve. The gradient flipping transition voltage/threshold voltage (Vₜₕ) in some implementations for the high twist cell 230 is low enough so that attenuation above Vₜₕ of the filter 200 reaches shade 9. In some implementations, to have a homogeneous mode at shade 9 and up to shade 13, the high twist cell 230 twist angle is higher than 110 degrees. In some implementations, to have a homogeneous mode at shade 9, the high twist cell 230 polarizers are offset by up to +−10 degrees.

In some implementation, in the low twist cell 210 the two residual LC layers (e.g., layers 115 in FIG. 1) with a positive A-plate effect do not cancel each other and act as a positive birefringence A-plate with the mean optical axis aligned along the vertical symmetry axis. The high twist cell 230 has a net effect of a positive birefringence A-plate aligned perpendicular to the vertical symmetry axis. The resulting symmetry of the A-plate effect in both cells 210, 230 results in an increased wide-viewing characteristic in the horizontal plane at any applied voltage in the driving range of the respective drivers (e.g., driver 110). The horizontal wide viewing is also very insensitive to the retardation values of the retarders and can work reasonably well even without retarders R. In some implementations, the retarders contribute to wide viewing at high shade values, whereas for low shade values, the negative C-plates contributes (e.g., only or primarily) to wide viewing in the horizontal plane, but the viewing in vertical plane is (or can be) weaker with retarders.

To increase the viewing angle in the vertical plane, in some implementations, polarizers need to be offset (e.g., from the +−45 degree crossed orientation) by several degrees. However, to reach class 1, very high voltages need to be applied (or very high dielectric anisotropy of the LC molecules must be used). To lower the driving voltages and increase the viewing in the vertical plane a biaxial film/layer can be used for compensation, as described below.

In some implementation the LC properties (e.g., dielectric anisotropy), for example, can be set so that both cells 210, 230 work in tandem and are complementary to each other. In this mode both cells 210, 230 can be driven by equal variable voltage devices (e.g., by drivers 110 or by one driver 110 could to both cells 210, 230) to voltages above the threshold voltage to operate in the homogeneous mode. In some implementations, cell 230 is driven above the threshold voltage and cell 210 is driven to any voltage (e.g., in the range of the drivers 110, which can be below, equal to or above the threshold voltage) to operate in the homogeneous mode. In this mode, the voltage applied to the second cell (e.g., low twist cell 210) can be used to adjust the attenuation of the filter 200. When the gradient-reversing cell (e.g., cell 230) is driven at a voltage below the threshold, the filter 200 operates in a heterogeneous mode.

In some implementations regarding filter 200, the first LC cell (e.g., 230) has polarizer transmission axes of the first top and bottom polarizers (e.g., 102) that are substantially parallel to LC alignment directions of the respective first top and bottom plates (e.g., 104) (e.g., in the extraordinary mode) and the second LC cell (e.g., 210) has polarizer transmission axes of the second top and bottom polarizers (e.g., 102) that are substantially perpendicular to LC alignment directions of the respective second top and bottom plates (e.g., 104) (e.g., in the ordinary mode).

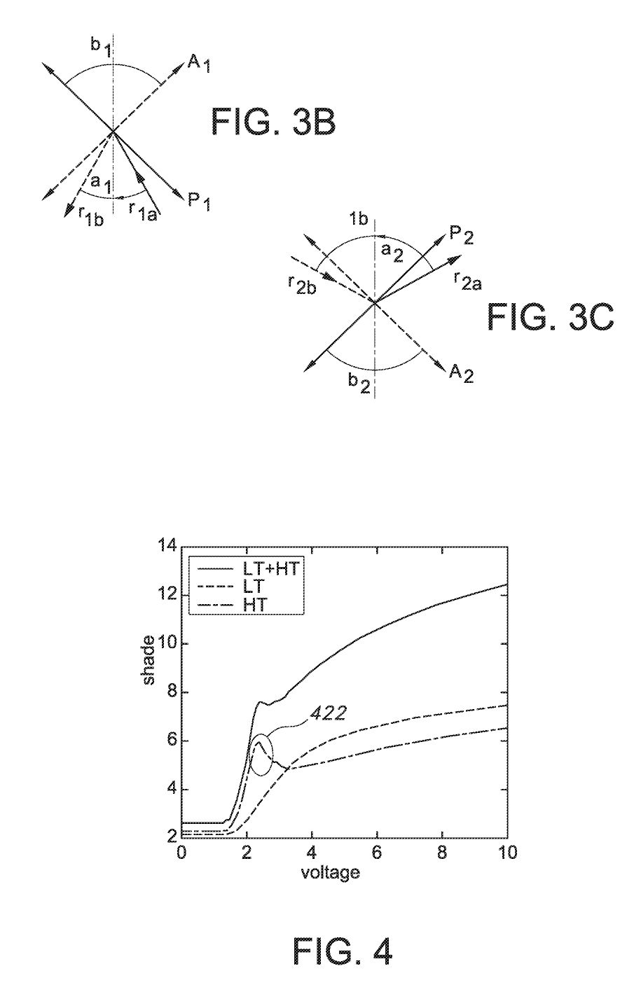

FIG. 3A is a block diagram of another example two-mode filter 300 having a low twist LC cell (top) 310 (e.g., second LC cell) and high twist LC cell (bottom) 330 (e.g., first LC cell). FIG. 3B is a representation of the orientation relationships of the low twist LC cell 310 of FIG. 3A, and FIG. 3C is a representation of the orientation relationships of the high twist LC cell 330 of FIG. 3A. In some implementations, regarding, filter 300, the first LC cell (330) has polarizer transmission axes of the first top and bottom polarizers (e.g., 102) that are substantially parallel to LC alignment directions of the respective first top and bottom plates (e.g., 104) and the second LC cell (e.g., 310) has polarizer transmission axes of the second top and bottom polarizers (e.g., 102) that are substantially parallel to LC alignment directions of the respective second top and bottom plates (e.g., 104).

FIG. 4 is a representation of attenuation/shade-voltage curves of a high twist cell 230, a low twist cell 210 and a two-cell filter 200 of the low and high twist cells (e.g., without the UV/IR filter). The high twist cell 230 has a kink 422 in its curve which corresponds to the gradient-reversing transition (e.g., threshold) voltage, which can be a range of voltages or a single voltage. When operating in a homogeneous mode, in some implementations, both cells 230 and 210 are driven at equal voltages above the threshold voltage. As described above, when the filter 200 is operating in the heterogeneous mode, the high twist cell 230 is set at or below the threshold voltage, and the low twist cell 210 can be used to set and adjust the attenuation (e.g., shade) of the filter 200.

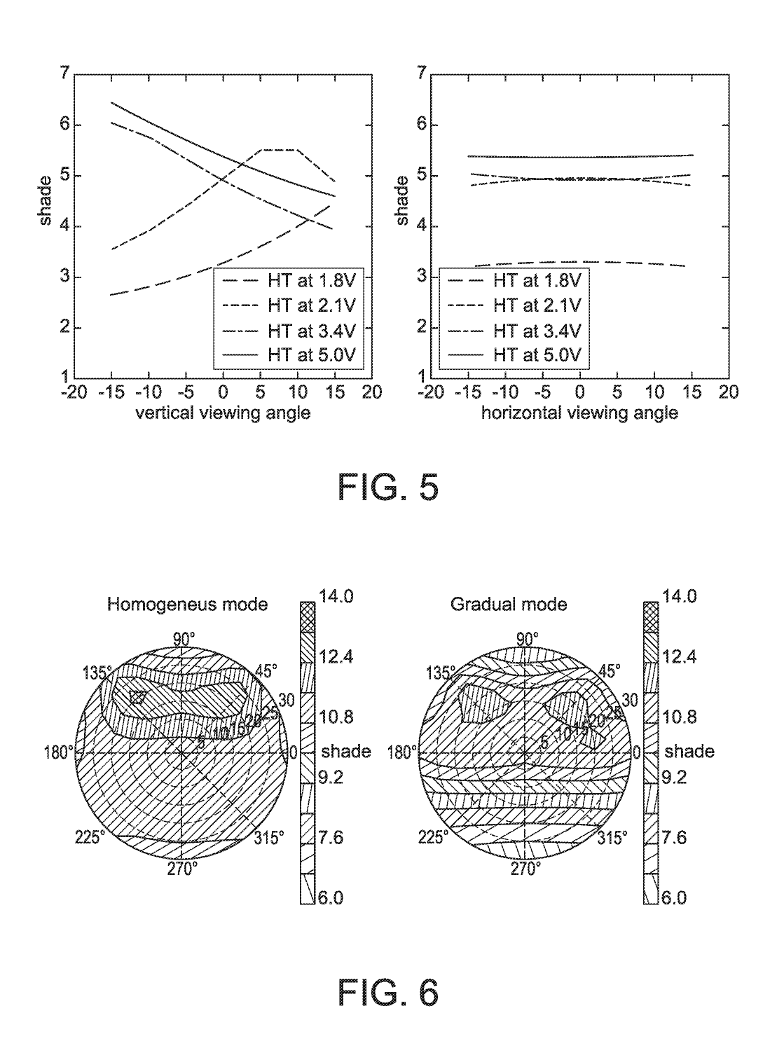

FIG. 5 is a representation of attenuation/shade-viewing angle curves of the high twist LC cell 230 of the two mode filter 200. For example, this high twist LC cell has 120 degrees of twist. As illustrated in the vertical viewing angle graph at 2.1V, the high twist LC cell constantly increases its shade up to about 5 degrees (to a shade of about 5.5), maintains that shade to about 10 degrees, and then decreases its shade above 10 degrees of vertical viewing angle. Thus, 2.1V represents the threshold/gradient flipping voltage of the high twist cell. This allows, for example, a welder using a welding mask with an ADF made of this two-mode filter operating in the heterogeneous mode to change the vertical shade by vertically tilting the mask up and down. The horizontal viewing angle graph shows that the high twist cell maintains a relatively flat and homogeneous shade at any of the applied voltages and that varying the voltage changes the level of shade/attenuation.

FIG. 6 is a representation of viewing angle properties of the two mode filter 200 where the low twist and high twist cells are symmetrically compensated with negative C retarders. Because the low-twist and high twist cells have complementary viewing angle transmission properties in the high voltage regime, wide and monotonous viewing can be obtained in the vertical direction.

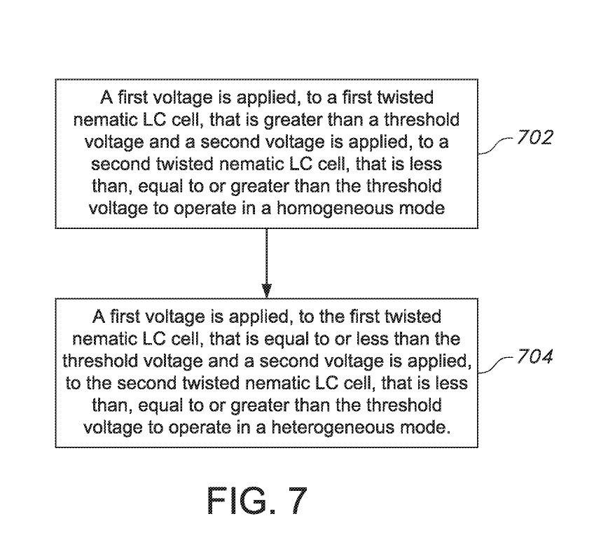

FIG. 7 is a flow chart of an example process for controlling a two-mode filter.

A first voltage is applied, to a first twisted nematic LC cell, that is greater than a threshold voltage and a second voltage is applied, to a second twisted nematic LC cell, that is less than, equal to or greater than the threshold voltage to operate in a homogeneous mode (702). For example, a first driver (e.g., a first driver 110) applies a voltage that is greater than a threshold voltage to the first LC cell (e.g., cell 230) and a second driver (e.g., a second driver 110) applies a voltage that is less than, equal to or greater than the threshold voltage to the second LC cell (e.g., cell 210) to operate in a homogeneous mode.

A first voltage is applied, to the first twisted nematic LC cell, that is equal to or less than the threshold voltage and a second voltage is applied, to the second twisted nematic LC cell, that is less than, equal to or greater than the threshold voltage to operate in a heterogeneous mode (704). For example, the first driver applies, to the first twisted nematic LC cell (e.g., cell 230), a voltage that is equal to or less than the threshold voltage and the second driver applies, to the second twisted nematic LC cell (e.g., cell 210), a voltage that is less than, equal to or greater than the threshold voltage to operate in a heterogeneous mode.

In some implementations, the first twisted nematic LC cell (e.g., 230) includes first top and bottom plates (e.g., 104) bounding liquid crystal material and has a twist angle of greater than 90 degrees and is configured to reverse its transmission characteristic at the threshold voltage across the first top and first bottom plates. In some implementations, the first top and bottom plates are bounded between first top and bottom polarizers (e.g., 102). In some implementations, the second twisted nematic LC cell (e.g., 210) includes second top and bottom plates (e.g., 104) bounding liquid crystal material and has a twist angle of less than 90 degrees. In some implementations, the second top and bottom plates are bounded between second top and bottom polarizers (e.g., 102).

As described above, other configurations of two-mode filters suitable for use in flat or curved ADFs (or other applications) are possible. Generally, if a C plate is stretched it gains a positive A-plate effect along the stretching direction and becomes a biaxial layer (e.g., film). The amount of in-plane retardation RO can be used to cancel the residual birefringence of both high twist and low twist cells at a maximum or high applied voltage. But, at lower voltages the A-plate retardation is not completely canceled, so it still behaves (optically) like a standard high twist or low twist cell, albeit with an effectively higher or lower twist angles, which is possible in part due to the residual A-plate birefringence of the LC cell being voltage dependent. When voltage is increased, the central part of the cell with homeotropic LC alignment becomes thicker while the residual layer near the cell surface becomes thinner. If a stretched film is put in place of the retarders R in, for example, FIG. 2A or 3A, with stretching directions along the symmetry axis for high twist cell, and perpendicular to the symmetry axis for low twist cell, then the A-plate retardation of the film can cancel the residual retardation of the LC at a predefined maximum voltage. At lower voltages, where the residual A-plate retardation of the LC cell increases, the A-plate compensated low twist or high twist cell behaves similar to a standard low twist or high twist cell with an effectively higher (or lower) twist angle. Thus, it is possible to utilize even lower twist angles in the low twist cell and higher twist angles of the high twist cell to increase the wide viewing in the low attenuation values at, for example, shades 9-11 and to decrease the driving voltage for the high attenuation (e.g., shade 13).

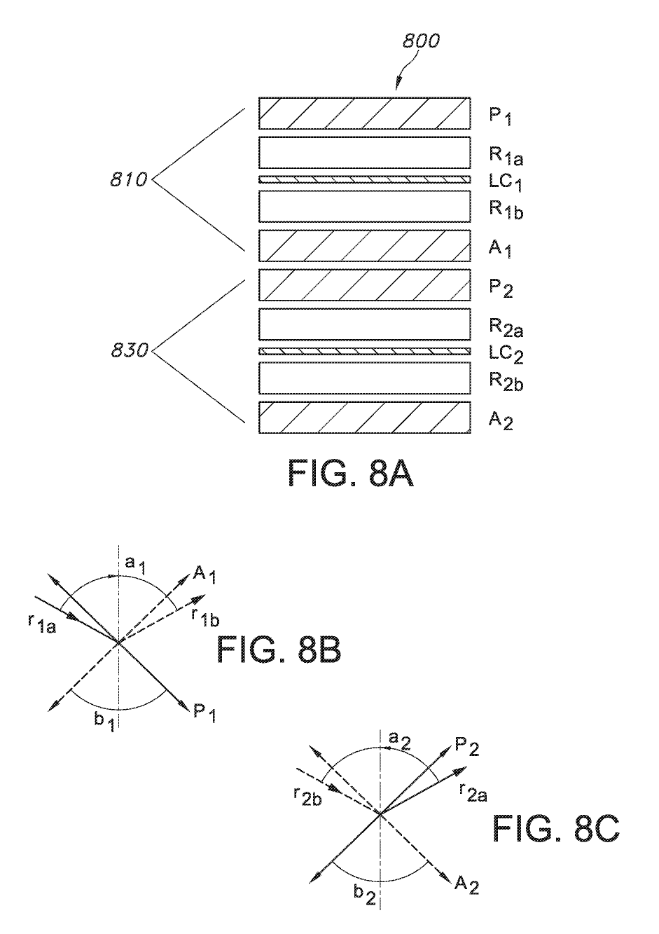

In some implementations, two high twist cells, e.g., with twist angles of between 120-180 degrees, can be used to form the two-mode filter. High twist cells have a greater symmetry, which results in a more homogeneous viewing angle properties. The gradient-flipping property, however, decreases with the increase of the twist angle. The optical configuration is shown in FIG. 8. FIG. 8A is a block diagram of an example two-mode filter 800 having a first high twist LC cell (top) 810 and a second high twist LC cell (bottom) 830 (e.g., symmetrically compensated with a stretched negative C retarders). FIG. 8B is a representation of the orientation relationships of the first high twist LC cell 810 of FIG. 8A, and FIG. 8C is a representation of the orientation relationships of the second high twist LC cell 830 of FIG. 8A.

In some implementations, the retarders are stretched uniaxial negative C plates, where the stretching direction (e.g., the direction of the A-plate optical axis) is aligned along the vertical symmetry axis. In some implementations, a single retarded can be used on each cell, and can be placed symmetrically with respect to each other. In some implementations, the negative C retardation is matched with the in-plane retardation of the LC cell at, for example, an intermediate voltage at shade 11. In some implementations, in the homogeneous mode, shades 9-13 are set at voltages above the Vₜₕ value in the low-slope regime (e.g., regime of the attenuation-voltage curve), while shades, for example, 5-8 can be set by applying voltage below the Vₜₕ in the steep-slope regime. In some implementations of two high twist cells, if a single retarder is used, the bisector of the LC alignment directions, and the retarder stretching direction are offset from the bisector of the two polarizers. This offset angle is in some implementations up to 15 degrees.

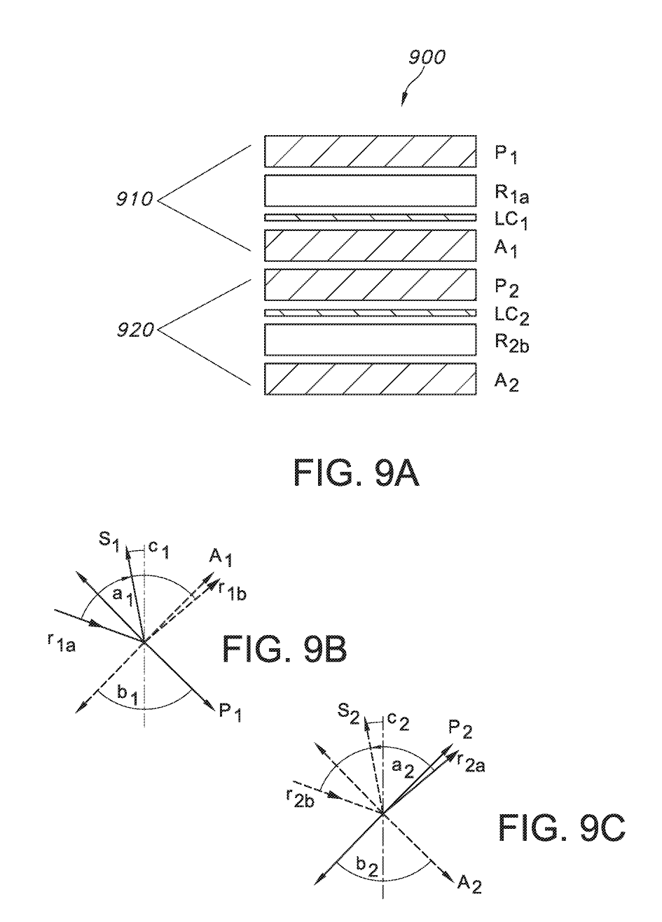

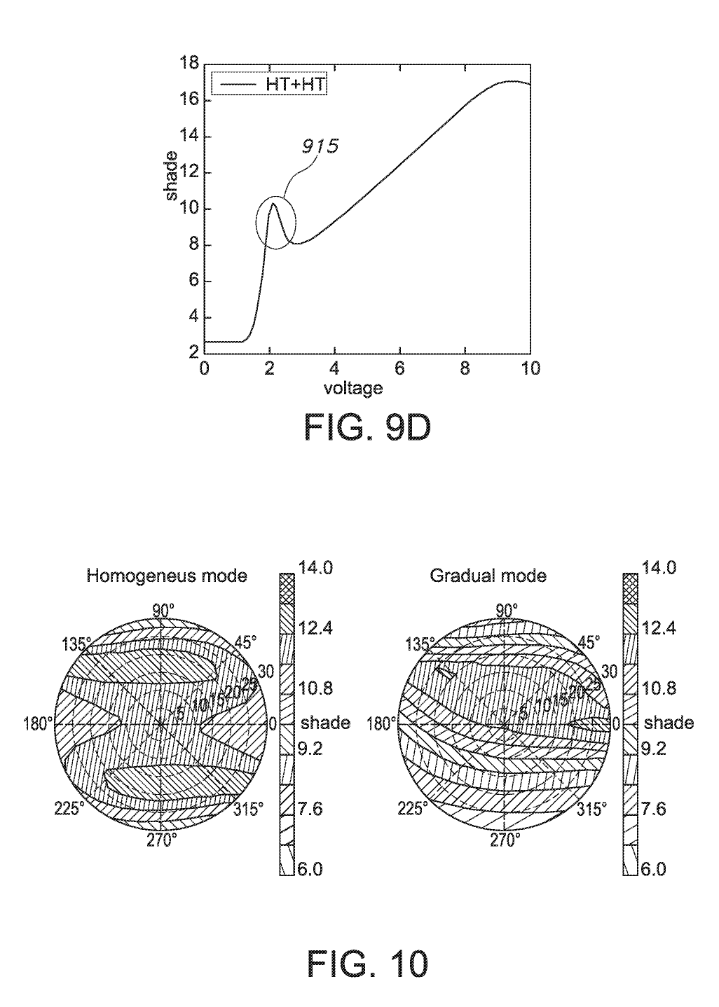

FIG. 9A is a block diagram representation of a two-mode filter 900 with high twist cell 910 and high twist cell 920 (e.g., compensated with a single stretched negative C retarder). FIG. 9B is a representation of the orientation relationships of the first high twist LC cell 910 of FIG. 9A, and FIG. 9C is a representation of the orientation relationships of the second high twist LC cell 930 of FIG. 9A. s1 and s2 are the orientations of the retarder stretching direction (a-plate orientation). C1 and c2 are the offset angles of the orientation of the retarder stretching directions (a-plate orientation) and the LC alignment symmetry axes s1 and s2 from the vertical symmetry axis. FIG. 9D is a representation of attenuation/shade-voltage curve of a two-cell filter 900 of the two high twist cells (e.g., 910 and 920) of FIG. 9A. The curve has a kink 915 that corresponds to the gradient reversing/transition voltage. When operating in a homogeneous mode, both high twist cells are driven at voltages above the threshold voltage. When the filter 900 is configured for a heterogeneous mode, one cell is set at or below the threshold voltage, and the voltage applied other cell is used to set the attenuation.

FIG. 10 is a representation of viewing angle properties of the two mode filter 900 with the two high twist cells (e.g., 910 and 920) (e.g., compensated with a single stretched negative C retarder). Because both cells have a complementary viewing angle transmission properties in the high voltage regime, a wide viewing can be obtained in the vertical direction.

Other two-mode filter configurations are all envisioned such as, in a low twist-high twist filter (e.g., filter 200), instead of the high twist cell, a 90 degree twist cell is used that is compensated with a biaxial film. The amount of in-plane RO retardation should be tuned to modify the 90 degree cell behavior to be gradient-flipping. If the low twist cell is compensated with a biaxial film the amount of in-plane RO retardation should be tuned to compensate the residual birefringence of the low twist cell, which allows the twist of the low twist cell to be even smaller (e.g., 0-30 degrees).

In a low twist-high twist filter, instead of the high twist cell, a 90 degree twist cell is used that is compensated with a biaxial film and instead of the low twist cell a 180 degree twisted cell is used compensated with a biaxial retarder. The amount of in-plane RO retardation should be tuned to modify the 90 degree cell behavior to be gradient-flipping and to compensate the residual birefringence of the 180 degree twisted cell cell.

In some implementations, two identical high twist cells (e.g., twist angles of around 130-140) are combined with biaxial retarders to form the two-mode filter. Here the amount of in-plane RO retardation is tuned to compensate the residual birefringence of the high-twist cell. Both cells work as a gradient-flipping, and both can be driven with an identical high voltage above the threshold voltage for the homogeneous operation. In the heterogeneous mode, one of the cells is driven at a low voltage (below the threshold) and one at a high voltage. The direction of the gradient (whether it is darker in the upper or lower vertical viewing angle) can be flipped here, depending on which of the two cells is driven at a low voltage.

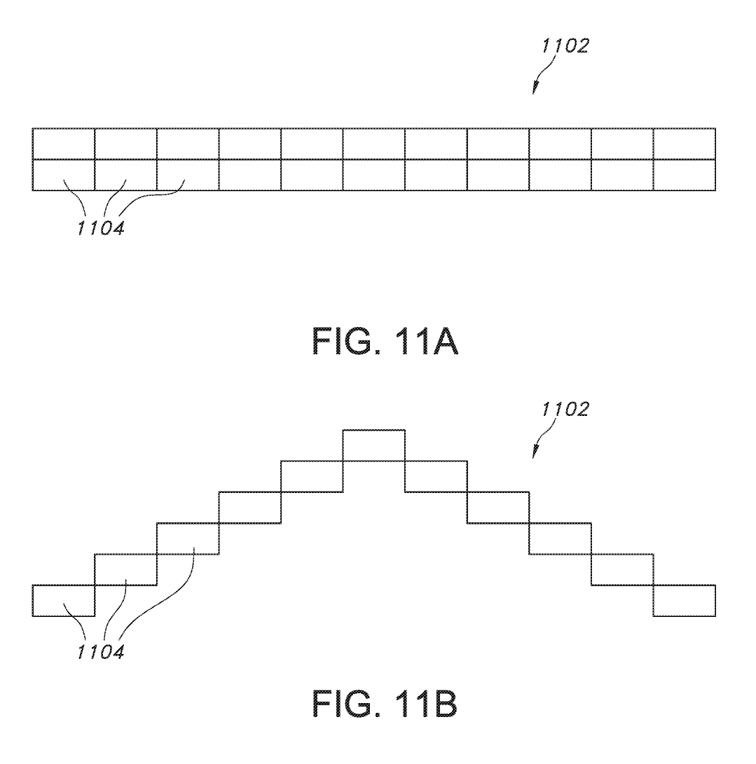

FIGS. 11A and 11B are block representations of a cylindrically curved filter surface 1102. FIG. 11A shows a top view of adjacent filters 1104 (e.g., 200 or 400, and/or as described in [007]) that can be used to form a curved (e.g., spherically or cylindrically) filter surface 1102. FIG. 11B is a side view of the surface 1102 showing the vertically offset filters 1104.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

This written description does not limit the invention to the precise terms set forth. Thus, while the invention has been described in detail with reference to the examples set forth above, those of ordinary skill in the art may effect alterations, modifications and variations to the examples without departing from the scope of the invention.

1. A two-mode filter device comprising:
A first twisted nematic liquid crystal (LC) cell comprising first top and bottom plates bounding liquid crystal material and having a twist angle of greater than 90 degrees and configured to reverse its transmission characteristic at a threshold voltage, wherein the first top and bottom plates are bounded between first top and bottom polarizers;
a second twisted nematic LC cell comprising second top and bottom plates bounding liquid crystal material and having a twist angle of less than 90 degrees, wherein the second top and bottom plates are bounded between second top and bottom polarizers;
a first driver configured to apply a first voltage across the first twisted nematic LC cell;
a second driver configured to apply a second voltage across the second twisted nematic LC cell; and
a controller configured to cause:
the first driver to apply the first voltage that is greater than the threshold voltage and the second driver to apply the second voltage that is less than, equal to or greater than the threshold voltage to operate in a homogeneous mode; and
the first driver to apply the first voltage that is equal to or less than the threshold voltage and the second driver to apply the second voltage that is less than, equal to or greater than the threshold voltage to operate in a heterogeneous mode.

2. The two-mode filter device of claim 1 comprising a plurality of cell units, wherein at least one cell unit comprises the first twisted nematic LC cell vertically stacked with the second twisted nematic LC cell, and further wherein the at least one cell unit is arranged adjacent to a cell unit to define a curved filter surface of the two-mode filter device.

3. The two-mode filter device of claim 1, wherein the first top and bottom plates and the second top and bottom plates are negative birefringent c-plates having a nominal negative birefringence with their optic axis oriented perpendicularly to the plates.

4. The two-mode filter device of claim 1, wherein the first top and bottom plates and the second top and bottom plates comprise plastic.

5. The two-mode filter device of claim 1, wherein the first top and bottom plates and the second top and bottom plates comprise glass and where two additional birefringent layers that are negative birefringent c-plates with optical axes aligned along the plates normal and with a negative birefringence are between the first top polarizer and first top plate and between the first bottom plate and first bottom polarizer, respectively.

6. The two-mode filter device of claim 5, wherein a total absolute value of the out-of-plane retardation introduced by the birefringent layers and the polarizers is between 200 and 400 nm for the first twisted nematic LC cell and less than 300 nm for the second twisted nematic LC cell.

7. The two-mode filter device of claim 1, wherein the twist angle of the first twisted nematic LC cell is in the range of 100 to 140 degrees and the twist angle of the second twisted nematic LC cell is in the range of 60 to 80 degrees.

8. The two-mode filter device of claim 1, wherein the transmission characteristic is a transmission gradient in a vertical plane which is along a vertical symmetry axis.

9. The two-mode filter device of claim 1, wherein the homogenous mode defines homogeneous attenuation in the horizontal and vertical planes, and wherein homogeneous attention in the vertical plane specifies a ratio of the luminous transmittance values measured for any angle of incidence up to +−15 degrees in a vertical direction and the transmittance value at normal incidence (or its reciprocal, whichever is greater) is less than 7.20 and homogeneous attention in the horizontal plane specifies a ratio of the luminous transmittance values measured for any angle of incidence up to +−15 degrees in a horizontal direction and the transmittance value at normal incidence (or its reciprocal, whichever is greater) be less than 2.68.

10. The two-mode filter device of claim 9, wherein the heterogeneous mode defines the homogeneous attenuation in the horizontal plane and inhomogeneous attenuation in the vertical plane, wherein inhomogeneous attenuation in the vertical plane specifies a ratio of the luminous transmittance values measured for any angle of incidence up to +−15 degrees in a vertical direction and the transmittance value at normal incidence (or its reciprocal, whichever is greater) is less than than 19.31.

11. The two-mode filter device of claim 1, wherein a bisector of polarization orientations of the first top and first bottom polarizers and a bisector of polarization orientations of the second top and bottom polarizers are aligned along a vertical symmetry axis and LC alignment layers are oriented symmetrically with respect to the vertical symmetry axis.

12. A method comprising:
applying a first voltage, to a first twisted nematic LC cell, that is greater than a threshold voltage and applying a second voltage to, a second twisted nematic LC cell, that is less than, equal to or greater than the threshold voltage to operate in a homogeneous mode; and
applying the first voltage, to the first twisted nematic LC cell, that is equal to or less than the threshold voltage and applying the second voltage, to the second twisted nematic LC cell, that is less than, equal to or greater than the threshold voltage to operate in a heterogeneous mode; and
wherein:
the first twisted nematic LC cell comprises first top and bottom plates bounding liquid crystal material and having a twist angle of greater than 90 degrees and configured to reverse its transmission characteristic at the threshold voltage across the first top and bottom plates, wherein the first top and bottom plates are bounded between first top and bottom polarizers; and
the second twisted nematic LC cell comprises second top and bottom plates bounding liquid crystal material and having a twist angle of less than 90 degrees, wherein the second top and bottom plates are bounded between second top and bottom polarizers.

13. The method of claim 12, wherein the first top and bottom plates and the second top and bottom plates are negative birefringent c-plates having a nominal negative birefringence with their optic axis oriented perpendicularly to the plates.

14. The method of claim 12, wherein the twist angle of the first twisted nematic LC cell is in the range of 100 to 140 degrees and the twist angle of the second twisted nematic LC cell is in the range of 60 to 80 degrees.

15. The method of claim 12, wherein the homogenous mode defines homogeneous attenuation in the horizontal and vertical planes, and wherein homogeneous attention in the vertical plane specifies ratio of the luminous transmittance values measured for any angle of incidence up to +−15 degrees in a vertical direction and the transmittance value at normal incidence (or its reciprocal, whichever is greater) be less than 7.20 and homogeneous attention in the horizontal plane specifies a ratio of the luminous transmittance values measured for any angle of incidence up to +−15 degrees in a horizontal direction and the transmittance value at normal incidence (or its reciprocal, whichever is greater) is less than 2.68.

16. The method of claim 12, wherein the heterogeneous mode defines the homogeneous attenuation in the horizontal plane and inhomogeneous attenuation in the vertical plane, wherein inhomogeneous attenuation in the vertical plane specifies a ratio of the luminous transmittance values measured for any angle of incidence up to +−15 degrees in a vertical direction and the transmittance value at normal incidence (or its reciprocal, whichever is greater) is less than be less than 19.31.

17. The method of claim 12, wherein the transmission characteristic is a transmission gradient in a vertical plane which is along a vertical symmetry axis.

18. The method of claim 12, wherein the first twisted nematic LC cell has polarizer absorption axes of the first top and bottom polarizers that are substantially perpendicular to LC alignment directions of the respective first top and bottom plates and the second twisted nematic LC has polarizer absorption axes of the second top and bottom polarizers that are substantially parallel to LC alignment directions of the respective second top and bottom plates.

19. The method of claim 12, wherein the first twisted nematic LC cell has polarizer absorption axes of the first top and bottom polarizers that are substantially perpendicular to LC alignment directions of the respective first top and bottom plates and the second twisted nematic LC has polarizer absorption axes of the second top and bottom polarizers that are substantially perpendicular to LC alignment directions of the respective second top and bottom plates.
